(19) United States
(12) Patent Application Publication
(10) Pub. No.: US 2024/0032811 A1
(43) Pub. Date: Feb. 1, 2024

(54) INDICATOR TEMPERATURE COMPENSATION IN FLOW RATE CALCULATION

(71) Applicant: Transonic Systems Inc., Ithaca, NY (US)

(72) Inventors: Nikolai M. Krivitski, Ithaca, NY (US); Emily Cirulnick, Ithaca, NY (US)

(21) Appl. No.: 18/227,615

(22) Filed: Jul. 28, 2023

Related U.S. Application Data
(60) Provisional application No. 63/393,106, filed on Jul. 28, 2022.

(51) Int. Cl. A61B 5/0275; A61B 5/0205; A61B 5/00

(52) U.S. Cl. CPC A61B 5/0275; A61B 5/02055; A61B 5/6852

(57) ABSTRACT

A catheter and introducer sheath assembly is provided for measuring a flow rate in a conduit by dilution methods. The catheter is configured to engage the introducer sheath in a predetermined alignment, where an indicator is passed through a volume defined by an exterior of the catheter and an interior of the introducer sheath to be introduced into the flow to be measured. The determination of the indicator temperature is extrapolated or estimated from (i) an observed temperature curve of an indicator sensor on the catheter that records a substitution of warmed fluid in the sheath by the introduced indicator and (ii) a pre-examined, predetermined or identified thermal properties or characteristics of the sensor, the catheter, or the sensor and catheter. The determination of the flow rate in the conduit is estimated using (i) the area of the dilution bolus and (ii) the return to baseline of the dilution bolus.

### CROSS-REFERENCE TO RELATED APPLICATIONS

The present application claims the benefit of US provisional patent application 63/393,106 filed Jul. 28, 2022, entitled CATHETER AND INTRODUCER SHEATH ASSEMBLY, the entire disclosure of which is hereby incorporated by reference.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO A SEQUENCE LISTING

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the invention

The present disclosure relates to calculating a flow rate of a flow in a conduit with a dilution indicator and particularly to calculating the flow rate based on only a portion of a measured dilution curve, and more particularly to calculating the flow rate from a portion of a dilution curve, wherein the dilution curve includes a portion measured during the introduction of the indicator to the flow to be measured. The present disclosure further provides for calculating the flow rate of the flow in the conduit, wherein the catheter is partially disposed within an introducer sheath, the catheter having a first sensor within an overlapping length of the introducer sheath and a second sensor beyond a terminal end of the introducer sheath and exposed to the flow in the conduit, wherein a controller is connected to the sensors and is configured to calculate the flow rate in the conduit.

It is contemplated the present disclosure can be employed in calculating a blood flow rate by indicator dilution techniques with the catheter, and more particularly to a method and apparatus for calculating the blood flow rate from dilution curves obtained by thermodilution sensors on the catheter, wherein the sensors are located at predetermined positions relative to an introducer sheath, and further wherein the indicator passes through a volume between an exterior of the catheter and the interior of the introducer sheath and into the blood flow being measured.

### 2. Description of Related Art

Blood flow measurement provides useful physiological information in biological systems. For example, it is useful to know the blood flow when an A-V fistulae or graft is first created to see if the procedure was successful. Additionally, the existence of a stenosis in the access typically requires an intervention to restore sufficient blood flow. Commonly, angioplasty is used to restore flow through the access and the flow needs to be monitored to determine the procedure was successful.

In one configuration, during an angioplasty procedure, the catheter and introducer sheath assembly is utilized as follows. The introducer sheath is introduced into a conduit, for example, a blocked coronary artery, in a patient's arm, neck or groin. A thin, long catheter having a tiny balloon at its tip is then inserted through the sheath. When the catheter is in position, the balloon is inflated at the area of stenosis. Blood flow measurements may be taken after the artery is open to determine whether the artery is sufficiently open. When the artery is sufficiently open, the catheter will be removed. The sheath may be maintained in the patient for several hours until the presence of blood-thinning medication administered during the procedure has decreased.

Thermodilution methods are used to measure blood flow in A-V shunts. This requires inserting a thermodilution catheter into the shunt through a sheath. Current thermodilution catheters are multi-lumen to accommodate thermistors and lumens for injections. The thermodilution catheter typically includes an injectate thermistor that measures temperature of injections—usually normal saline at room temperature or cooled to below room temperature. A dilution thermistor measures the blood temperature before and during the indicator mixing with blood. A separate lumen for the indicator injection provides a passageway for saline into the blood stream required for flow measurements.

Such multi-lumen, small size, precise extrusion components, requiring the presence of an injection port and the necessity of a manifold, increase manufacturing costs and substantially decrease yields in mass production of such catheters. Thus, these catheters usually have a relatively large end user price.

Therefore, the need exists for a simple, relatively easy to manufacture catheter, thereby providing a low-cost catheter for blood flow measurement. A further need exists for a simple low-cost catheter for measuring blood flow to simplify the measurement of blood flow using a simple catheter and a standard sheath used in the procedure.

It is an object of the present disclosure to provide a simplified, inexpensive, and accurate flow measurement catheter and use of such catheter, and particularly in conjunction with an introducer sheath.

### BRIEF SUMMARY OF THE INVENTION

In one configuration, the present disclosure includes method of calculating a flow rate of a flow in a conduit based on an indicator introduced into the flow, the method including introducing an indicator into a flow in a conduit; measuring, by a sensor, a dilution curve in the conduit from the passing of the indicator in the conduit; identifying in the measured dilution curve, a portion of the dilution curve created after termination of the introducing the indicator into the flow; and calculating a flow rate in the conduit corresponding to the identified portion of the dilution curve.

An apparatus is disclosed including a catheter assembly for placement within a conduit having a flow with a flow rate to be calculated, the catheter assembly comprising (i) an introducer sheath having an exterior, an interior, and an introducing port, and (ii) a catheter having an exterior, wherein a first sensor is located on a distal end of the catheter and configured to generate a dilution curve; and a controller operably connected to the first sensor, the controller configured to calculate the flow rate in the flow in the conduit, wherein the calculated flow rate at least partly corresponds to the generated dilution curve.

A further apparatus is provided for calculating a flow rate of a flow in a conduit, the apparatus including a first sensor configured to measure a dilution curve in a flow in a conduit, the dilution curve at least partly concurrent with an introduction of an indicator into the flow; and a controller operably connected to the sensor, the controller configured to calculate a flow rate in the conduit, wherein the calculated flow rate at least partly corresponds to an identified portion of the dilution curve, the identified portion created after a termination of the introduction of the indicator into the flow.

### BRIEF DESCRIPTION OF THE SEVERAL VIEWS OF THE DRAWING(S)

FIG. 1 is a graphical representation of the indicator dilution catheter showing the location of the two thermal sensors and the visible marker.

FIG. 2 is the graphical representation of the indicator dilution catheter inserted into the introducer sheath in the vessel with the thermal indicator (injectate) sensor located in the extracorporeal region of the introducer sheath, where the thermal injectate sensor is located such that the injectate will pass over it.

FIG. 3 is the graphical representation of the indicator dilution catheter inserted into the introducer sheath in the vessel with the thermal indicator (injectate) sensor located in the intracorporeal region of the introducer sheath, where the thermal indicator (injectate) sensor is located such that the indicator will pass over it.

FIG. 4 is a graphical representation of the indicator dilution catheter inserted into an A-V graft with the thermal dilution sensor located in the AV graft and indicator injectate thermal sensor is in the extracorporeal region of the introducer sheath.

FIG. 5 is a graphical representation of the real versus sensed temperature at the thermal injectate sensor, wherein the extrapolated curve estimates the actual temperature of the introduced indicator.

FIG. 6 is a graphical representation of determining the time constant of the sensor, the catheter, or both the sensor and the catheter when moving from a steady warm temperature to a steady cold temperature.

FIG. 7 is a graphical representation of the dilution curve and parameters associated with the thermal curve along with the flow shown in the vessel due to the introduced indicator flow.

FIG. 8 is a graphical representation of the downslope of the thermal injectant curve related to the introduction or injection time.

FIG. 9 is the graphical representation of the rate of warming for different flow rates of conduit.

FIG. 10 is a flow chart of the method of calculating the flow rate in the conduit.

### DETAILED DESCRIPTION OF THE INVENTION

Generally, the present system provides for the measurement of a flow rate of a flow in a conduit 8. The flow is the movement of a fluid, such as a liquid, and the flow rate is the volume rate of the flow, that is the volume of fluid passing a given point or cross section in a given period time.

Although the present description is set forth in terms of calculating a blood rate in a vessel, and particularly an arterio-venous (A-V) shunt, it is understood the present system is not limited only to A-V shunts, but can be employed in any vessel, conduit or channel, where the amount of flow resistance and/or the location of the flow resistance in the flow path (relative to the injection site) is unknown. The conduit may be any of a variety of liquid conducting members including arteries, veins, heart chambers, shunts, vessels, tubes and lumens.

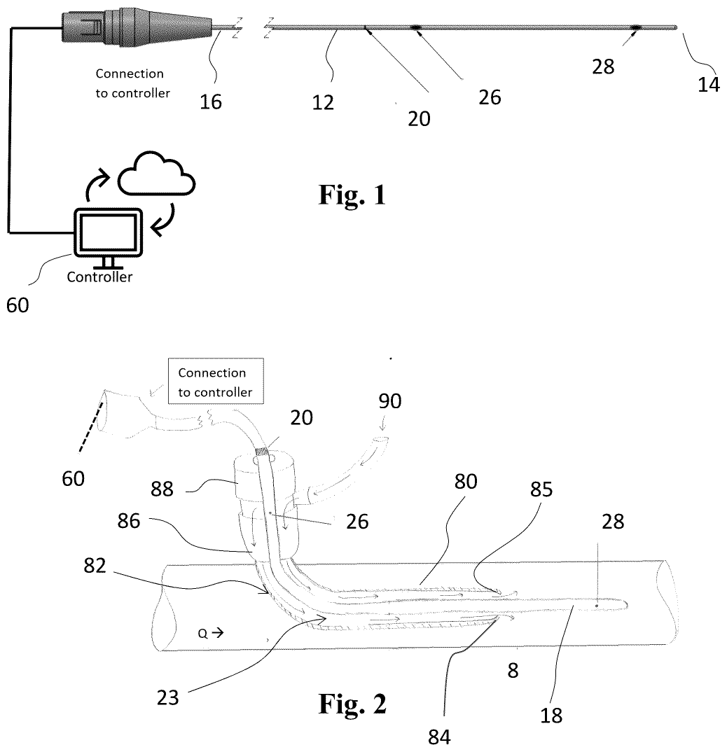

As shown in the figures, including FIG. 1, the present system includes a catheter 10 and a controller 60 operably connected to the catheter, wherein the catheter cooperatively engages an introducer sheath 80.

The introducer sheath 80 is a long, flexible tube that is inserted into the conduit 8, such as a vein or artery to provide a larger opening for the insertion of the catheter 10. The introducer sheath 80 includes a sheath body 82, a hub 88, and a side arm port 90, and can further include a dilator (not shown).

The sheath body 82 is the main part of the introducer sheath 80 and is elongate having a distal end 84 a distal port 85, and a proximal end 86. The sheath body 82 can include a sharp tip (not shown) that is configured to puncture the conduit, such as a blood vessel or other body cavity. The hub 88 is a connector attached to the proximal end 86 of the sheath body 82. The hub 88 includes at least one port 90, such as the side arm port configured to connect the introducer sheath 80 to other medical devices, such as catheters, guidewires, and syringes. The side arm port 90 is an opening located on the side of the sheath body 82 or the hub 88. The side arm port 90 can be used to inject fluids or medications into the blood vessel or other body cavity. The dilator is a hollow tube inserted through the sheath body and can be used to widen the opening in the conduit, the vessel or other body cavity, making it easier for the catheter to pass through.

In one configuration, the catheter 10 is an indicator dilution catheter having an elongate catheter body 12 including a distal end 14, a proximal end 16, and in one configuration, a single lumen 18 extending from the distal end to the proximal end. Although the present description is set forth in terms of a single lumen 18, it is understood the catheter body 12 can include a plurality of lumens extending from the distal end to the proximal end, wherein the plurality of lumens can have different cross-sectional areas, or each lumen can have the same cross-sectional area. The catheter body 12 further includes, a seating indicia 20, such as but not limited to a visible mark or print, or a detent or snap location.

When the catheter 10 is operably located with respect to the introducer sheath 80, a length of the catheter is disposed within a length of the introducer sheath, wherein a further length of the catheter extends through the distal port 85 and beyond the distal end 84 of the introducer sheath. A priming volume 23 is defined as a volume between an exterior of the length of catheter 10 that is located within the introducer sheath 80 and an inside of the introducer sheath retaining the catheter between the introduction port 90 of the introducer sheath and the distal end 84 of the introducer sheath.

The catheter 10 further includes a first sensor 26 towards the proximal end 16 of the catheter body 12 and a second sensor 28 intermediate the first sensor and the distal end 14 of the catheter body. In relation to the introducer sheath 80, the indicator (first) sensor 26 is located along the priming volume and the dilution (second) sensor 28 is beyond the distal end 84 of the introducer sheath 80 to be directly exposed to the flow in the conduit 8. That is, in one configuration, the catheter 10 includes the indicator sensor 26 and the dilution sensor 28, wherein the indicator sensor and the dilution sensor are located such that the indicator sensor is located within a portion of the introducer sheath 80 that is exposed to the indicator during introduction (or injection) of the indicator into the flow in the conduit 8 (FIGS. 1-4) and the dilution sensor 28 is located outside or beyond the distal end 84 of the introducer sheath 80.

The first and second sensors 26, 28 can be indicator sensors configured to measure the introduced indicator, such as dilution sensors, and in one configuration the first and second sensors are thermal sensors. As set forth below, in one configuration of the system, the sensors are set forth in terms of thermal dilution, however it is understood the sensors can be any sensor capable of measuring the introduced indicator.

The sensors are set forth as thermal sensors including thermistors or thermocouples, as well as any sensor that can measure temperature, electrical impedance sensor, ultrasound velocity sensor, and blood density sensor. The sensors are employed to detect passage of the indicator and thus measures, identified or monitors a blood parameter or property, and particularly variations of the blood parameter or property. Thus, the sensors are capable of sensing a change is a blood property, parameter or characteristic. For purposes of the disclosure, the sensors can be referred to as a dilution sensor, but this label is not intended to limit the scope of available sensors. Ultrasound velocity sensors as well as temperature sensors and optical sensors, density or electrical impedance sensors, chemical or physical sensors may be used to detect changes in blood parameters. It is understood that other sensors that can detect blood property changes may be employed. The operating parameters of the particular system will substantially dictate the specific design characteristics of the dilution sensor, such as the particular sound velocity sensor. For example, if a thermal sensor is employed, the thermal sensor can be any sensor that can measure temperature, for example, but not limited to thermistor, thermocouple, electrical impedance sensor (electrical impedance of blood changes with temperature change), ultrasound velocity sensor (blood ultrasound velocity changes with temperature), blood density sensor and analogous devices. Therefore, any type of optical sensor, impedance, resistance or electrical sensors which measure a changeable blood parameter such as the sound or ultrasound velocity in blood can be calibrated.

It is further contemplated, the sensors can also be sensors for sensing markers in the flow (or blood), including native or introduced particles that could be used as a surrogate. In select configurations, the sensors can measure different blood properties: such as but not limited to temperature, Doppler frequency, electrical impedance, optical properties, density, ultrasound velocity, concentration of glucose, oxygen saturation and other blood substances (any physical, electrical or chemical blood properties). For purposes of description, the present configuration is set forth in terms of thermal dilution and temperature signals.

For purposes of description, the first sensor 26 is set forth as an injectate or indicator sensor for sensing the temperature of the indicator, and the second sensor 28, is set forth as a dilution sensor, configured for detecting the passage of the introduced, such as injected, indicator in the flow to be measured in the conduit 8, such as a blood flow. That is, the second sensor 28 referred to as a dilution sensor is a sensor that senses a respective property or characteristic of the mixed or diluted flow in the conduit 8, and in the present configuration a temperature of flow in the conduit. By determining the flow rate in the conduit 8, it can be determined whether the flow rate is too fast or too slow from a desired flow. For example, in most well-functioning lower arm artificial grafts, the blood flow rate is in the range of 1000-1600 ml/min for a 1 inch diameter access. A calculated or determined flow rate that is too low, for example 300-500 ml/min, can indicate a narrowing or resistance in the access.

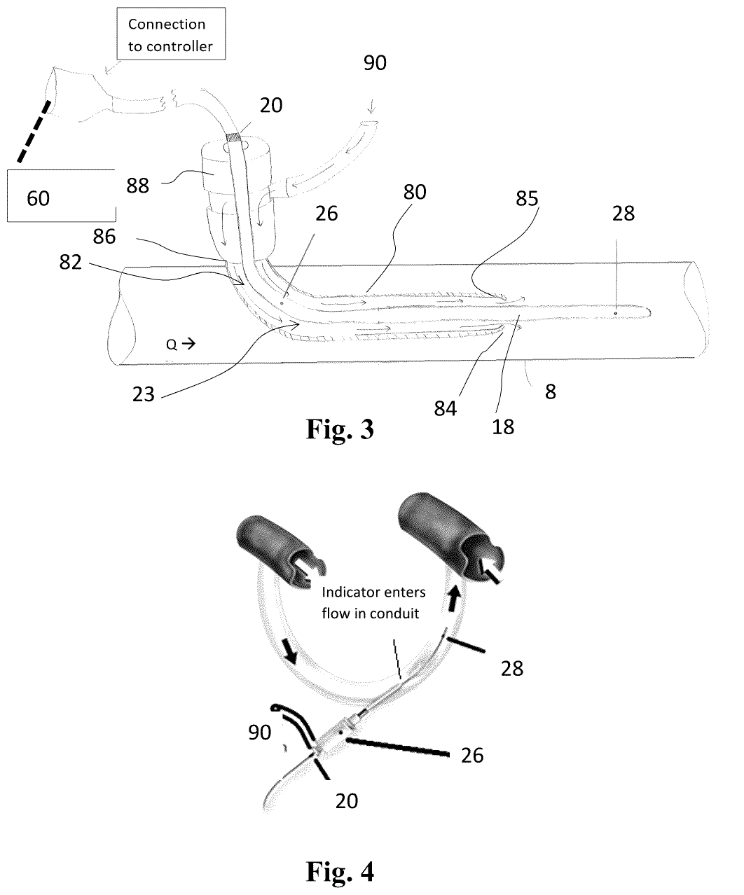

The indicator sensor 26 is located on the portion of the catheter body 12 that is within the introducer sheath 80 so that the injected indicator passes over the indicator sensor. It is understood the indicator sensor 26 can be in two separate locations in this configuration. The indicator sensor can either be within an extracorporeal portion 92 (FIG. 2) of the introducer sheath or an intravascular portion 94 of the introducer sheath (FIG. 3). The seating indicia 20, such as visible print, prevents the catheter 10 from being inserted so far into the introducer sheath 80 to cause the indicator sensor to be located outside the introducer sheath, and in the conduit 8. The seating indicia 20 further provides that the dilution thermal sensor 28 is located outside the introducer sheath 80 and in the conduit 8.

The dilution sensor 28 is located downstream on the catheter body 12 beyond the distal end 84 of the introducer sheath 80 and within the conduit 8, such as the A-V shunt or other vessel, and senses the dilution of the flowing fluid (blood) from the indicator. In one configuration, the indicator sensor 26 and the dilution sensor 28 are located on the catheter body 12 distal to the seating indicia 20, such as the visible print.

The indicator includes but is not limited to: blood hematocrit, blood protein, sodium chloride, dyes, blood urea nitrogen, a change in ultrafiltration rate, glucose, lithium chloride and radioactive isotopes and microspheres, or any other measurable blood property or parameter. An injectable indicator may be any of the known indicators including saline, electrolytes, water and temperature gradient indicator bolus. Preferably, the indicator is non toxic with respect to the patient and non reactive with the material of the system. The indicator may be any substance that will change a blood chemical or physical characteristic. The indicator may be a physically injected material such as saline. Alternatively, the indicator may be by manipulating blood properties without introduction of an indicator volume, such as by heating or cooling the blood or changing electromagnetic blood properties or chemical blood properties.

Referring to FIGS. 2, 3, and 4, the catheter is shown operably located in the conduit 8, as an arterio-venous (A-V) shunt. The A-V shunt has a blood flow shown by the direction of the arrow labelled Q. The A-V shunt has a single flow direction, where the blood flows from the arterial (upstream) side to the venous (downstream) side. Thus, the term downstream indicates the object is directed with the flow and upstream is proximal to the injection.

The catheter body 12 includes an extracorporeal portion 30 encompassing the proximal end 16 and an intracorporeal portion 32 encompassing the distal end 14. The extracorporeal portion 30 of the catheter body 12 is that portion of the catheter body that is not operably located within the body or the vessel, such as the shunt (or the conduit 8). The intracorporeal portion 32 of the catheter body 12 is that portion, or length that is located within the body or the vessel, such as the shunt (or conduit 8). That is, the extracorporeal portion 30 is the area outside the body (patient), and the intracorporeal portion 32 is the portion of the introducer sheath within the body (patient). In one configuration, a majority of the intracorporeal portion 32 is in contact with the blood flow in the shunt or conduit 8. As set forth above, the priming volume 23 is defined as the volume between the exterior of the length of catheter 10 that is located within the introducer sheath 80 and the inside of the introducer sheath retaining the catheter, generally between the introduction port 90 of the introducer sheath and the distal end of the introducer sheath 84.

The positioning of the catheter body 12 relative to the introducer sheath 80 is set by the seating indicia 20. For example, where the seating indicia 20 which is a visible mark, the catheter body 12 is aligned relative to the introducer sheath 80 by aligning the visible mark of the seating indicia with a determined corresponding mark, such as shown in FIG. 2.

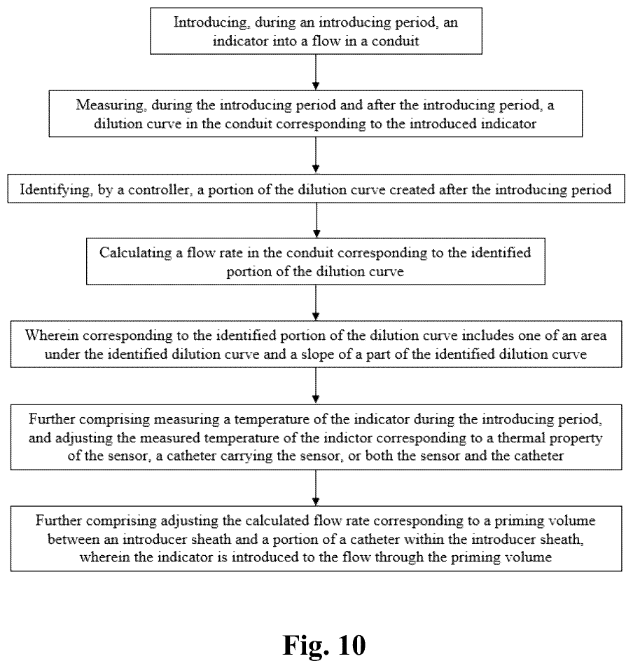

The sensors of the catheter are connected to the controller 60, wherein the controller includes a processor having hardware and software configured to implement the present algorithms, as set forth below and in FIG. 10, and can include local or remote memory.

The controller 60 is configured for determining or calculating the flow rate of the flow in the conduit 8 in response to the corresponding signals from the respective sensor(s) 26, 28 and the introduced indicator. The controller 60 can be any of a variety of devices including a computer employing software for performing the calculations, or a dedicated analog circuit device, or a calculation routine into which measured parameters are manually entered. That is, the controller 60 can be a stand-alone device such as a personal computer, a dedicated device or embedded in one of the components. The controller 60 may be connected to the sensors 26, 28 and configured to implement the equations as set forth herein.

In one configuration, the present disclosure provides a system and method of obtaining, including assessing or measuring, and collectively “calculating” the flow rate (such as the blood flow rate) of a flow in a conduit, and in one configuration, calculating the blood flow rate in an A-V access using thermodilution. A temperature signal, such as an indicator and more particularly an indicator having a lower temperature or a higher temperature than the flow of the flow rate to be calculated Q, is introduced into the flow at an upstream location and a downstream dilution signal is sensed.

$$Q = \frac{( {T}_{b} - {T}_{inj} ) k * {V}_{inj}}{S} \qquad Eq . 1$$

where Q—is the flow rate to be calculated, T_{b} is the initial temperature of the flow, Tᵢₙⱼ is the temperature of the indicator, k is a constant, wherein k is typically taken to be 1.08, Vᵢₙⱼ is the volume of indicator, and S is the area under the dilution curve recorded by a dilution sensor in the flow to be measured.

In a specific configuration, the flow rate to be measured is the blood flow rate in a conduit such as a vessel and is calculated using the Equation 1 where Q—is the blood flow rate,

T_{b} is the initial temperature of the blood flow, Tᵢₙⱼ is the temperature of the indicator, k is a constant, wherein k is typically taken to be 1.08, Vᵢₙⱼ is the volume of indicator, and S is the area under the dilution curve recorded by a dilution sensor in the flow to be measured.

In one configuration of the present system, the introducer sheath 80 is employed (i) to introduce the catheter 10 into the conduit 8, such as blood vessel, and (ii) to pass the indicator into the flow to be measured, such as the blood flow in the conduit 8. In one configuration, the introducer sheath 80 includes the side arm port 90, wherein the indicator is injected through the side arm port, thereby eliminating the need of an injection lumen in the catheter 10. However, it is recognized that the use of a portion of the dilution curve, as set forth below, can be employed without requiring use of the introducer sheath 80.

In the configuration having the introducer sheath 80, the present system is configured to address a number of complications associated with the introduction of the indicator through the introducer sheath, wherein the priming volume 23 exists between the catheter 10 and the introducer sheath. One complication in the present system is the existence of the priming volume 23 between the external surface of the catheter 10 and the internal surface of the introducer sheath 80. That is, a portion of the introduced indicator passing through the side arm port 90 will not pass into the flow to be measured as a portion of the indicator remains in the priming volume 23. A further complication is determining the temperature of the indicator (injectate). That is, as the indicator sensor 26 is disposed within the priming volume 23, and hence within the introducer sheath 80, the temperature of the indicator sensor trends towards the temperature of the flow in the conduit 8, thereby introducing error into the reading of the indicator sensor. For example, it is not uncommon for blood or other fluid to backflow into the introducer sheath 80 (and thus into the priming volume 23) and cause the indicator sensor 26 to read a higher temperature, even if the indicator sensor is located in the extracorporeal portion 30. A further complication is the influence of the added flow rate by the introduction of the indicator to the flow to be measured. That is, in the present system the indicator can be introduced through the introducer sheath 80 and the priming volume 23 at a higher rate than through the traditional catheter lumen. This higher rate of introduction can be sufficient to create a material change in the flow rate to the be calculated.

It is helpful to ensure that a correct volume of indicator will be used in Eq. 1. It is recognized that as a portion of the introduced indicator will remain in the introducer sheath 80, such as in the priming volume 23, the volume of indicator used in Eq. 1 needs to be adjusted. In a simple case, the decreased volume of the indicator (injectate) is the volume of the indicator that remains within the introducer sheath 80. For purposes of description, the volume of the indicator that remains within the introducer sheath 80 is taken as the priming volume 23, the volume between the exterior of the length of catheter that is located within the introducer sheath and the inside of the introducer sheath retaining the catheter. For example, this underdelivered volume, priming volume 23, is shown in (FIGS. 2 and 3).

The Eq. 1 can then be rewritten:

$$Q = \frac{[ ( {T}_{b} - ( {T}_{sh} - Δ {T}_{min} - Δ {T}_{ap} ) ] k * ( {V}_{inj} - {V}_{pr} )}{{S}_{inj}} \qquad Eq . 2$$

where Tₛₕ is the temperature of the fluid in the introducer sheath 80, such as in the priming volume 23, prior to the introduction of the indicator as recorded by indicator sensor 26 located inside the introducer sheath, ΔTₘᵢₙ—is the change in temperature measured by the indicator sensor at the end of injection at an apex of the dilution curve, Sᵢₙⱼ is the area under the dilution curve produced by indicator sensor, recorded by the dilution sensor, ΔTₐₚ—is an additional temperature decrease based on an approximation, extrapolation, estimation based on the recorded temperature, bench data, or a combination of both to provide value for the indicator temperature which is closer to an actual temperature of the indicator, Vₚᵣ is the volume of indicator that did not enter the flow to be calculated, such as a blood stream (which volume can be approximated as the priming volume, where the priming volume is the internal volume of the introducer sheath exposed to the indicator less the volume of the catheter 10 located within that internal volume of the introducer sheath).

During introduction, such as injection of the indicator, at least a portion of the fluid volume Vₚᵣ located between the exterior of the catheter 10 and interior walls of the introducer sheath 80 (the priming volume 23) with temperature Tₛₕ will also enter the flow to be measured, such as the blood flow and mix with the blood flow and create part of the recorded area under dilution curve S. In case of the injection volume Vᵢₙⱼ>>Vₚᵣ this influence may be negligible. However, in case in which these volumes are closer, for more accurate calculation of Q, these factors need to be considered. Taking that Tₛₕ,T_{b} and Vₚᵣ are known, then:

$$Q = \frac{[ ( {T}_{b} - {T}_{sh} ) ] k * {V}_{pr}}{{S}_{pr}} \qquad Eq . 3$$

Where Sₚᵣ is the area under the dilution curve produced by fluid located in the priming volume, as recorded by the dilution sensor.

Combining Eq. 2 and Eq. 3:

$$Q = \frac{\begin{matrix}[ ( {T}_{b} - ( {T}_{sh} - Δ {T}_{min} - Δ {T}_{ap} ) ] k * \\ ( {V}_{inj} - {V}_{pr} ) + [ ( {T}_{b} - {T}_{sh} ) ] k * {V}_{pr}\end{matrix}}{S} - \frac{{V}_{inj}}{2 * {t}_{inj}} , \qquad Eq . 4$$

Where S=Sₚᵣ+Sᵢₙⱼ is the area under the dilution curve recorded by the dilution sensor, tᵢₙⱼ—the time length of the injection, (which time can be approximated by the width of the recorded dilution curve, or from the shape of dilution (such as temperature) curve recorded by the indicator sensor).

The second term of Eq. 4 is added, to adjust the results of the flow measurements for the extra flow of the indicator introduced during introduction of the indicator.

The parameters that are known or can be measured in Eq. 4 include: T_{b}—the initial temperature of the flow in the conduit, such as the blood temperature, Tₛₕ—the temperature of the fluid in the introducer sheath 80 (in the priming volume 23), prior to the injection, Tₘᵢₙ—the temperature of the indicator sensor 26 at the end of the injection, S—the area under the dilution curve recorded by the dilution sensor 28, tᵢₙⱼ—the width of dilution curve, k—the flow constant, Vᵢₙⱼ—the volume of indicator, Vₚᵣ—the volume of the indicator that did not enter the flow to be measured (such as the blood flow) that is retained in the priming volume. The value of Tₐₚ—the additional temperature decrease from the recorded temperature to true indicator temperature is unknown. However, an approximation of Tₐₚ can be made by an extrapolation or estimation. For example, as set forth below, if the time constant of the catheter is known, then Tᵢₙⱼ can be predicted, from which Tₐₚ determined.

With the location of the indicator sensor 26 within the introducer sheath 80, it can be assumed the indicator sensor will be exposed to temperatures close to the flow (or blood) temperature in the conduit 8 before the introduction (such as the injection) of the indicator into the flow of the flow rate to the calculated. For example, a portion of the flow from the conduit 8 will occupy at least a portion of the priming volume 23, thereby warming the indicator sensor 26 towards the temperature of the flow in the conduit. Thus, there is a high likelihood that during an introduction period (the time period during which indicator is introduced into the flow to be measured) of the indicator (injectate), which may last for example 3-5 seconds, the indicator sensor 26 may not have sufficient time to reach the indicator temperature to provide an accurate reading. For example, if the indicator is a liquid cooled below the temperature of the flow in the conduit 8, then the indicator sensor 26 having been slightly warmed by the flow in the conduit, may not be cooled by the indicator to the actual temperature of the indicator (injectate) within the introduction period. This will introduce error in the calculation of the blood flow rate (Eq. 1). To reduce the error from an incorrect measurement of the temperature of the indicator (injectate), an approximation algorithm can be used.

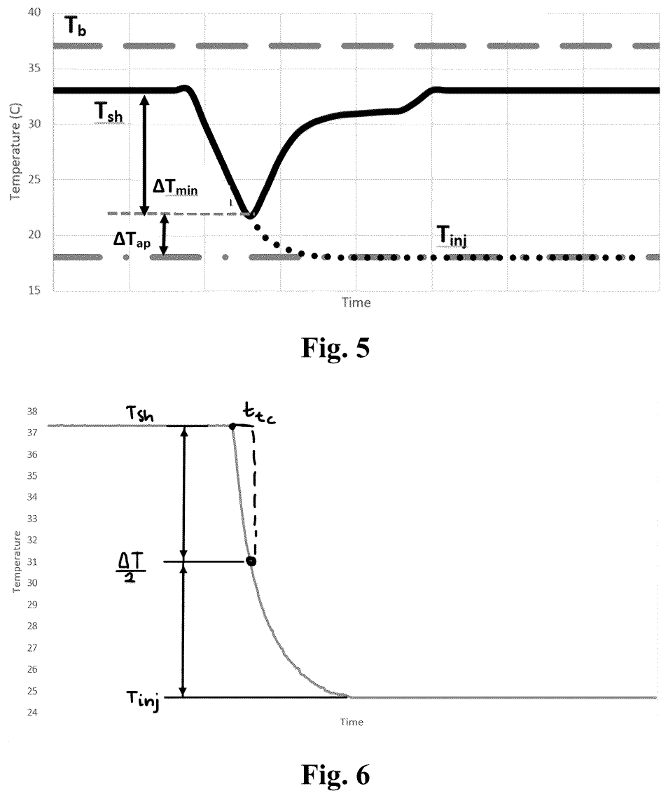

The present disclosure recognizes that in injections through the introducer sheath 80, the (thermal) indicator sensor 26 is incapable of sensing the true temperature of the indicator. For example, as the indicator sensor 26 approaches the A-V shunt, the indicator sensor warms due to its proximity to warmer blood. FIG. 5 shows the inability of the indicator sensor 26 to reach the same temperature of the indicator within the introduction period, when the indicator sensor is located within the intravascular areas of the introducer sheath and instead measures a temperature that is greater than the actual temperature of the indicator (for an indicator having an actual temperature less than the temperature of the flow to be measured, such as the blood temperature). While the indicator sensor 26 in the extracorporeal portion of the introducer sheath may also measure a temperature warmer than the actual indicator, the difference is usually less than the indicator sensor 26 located in the intravascular portion of the introducer sheath.

A higher (for indicators having a temperature lower than the flow to be measured) recorded temperature of the indicator by the indicator sensor 26 affects the resulting calculated flow by decreasing the numerator which decreases the overall calculated flow. The temperature read by the indicator sensor 26 is insufficient for calculating the proper flow as there is not sufficient time for the indicator sensor to cool to the reduced temperature of the indicator while the indicator quickly passes over the indicator sensor within the introduction period. It is, therefore, advantageous to estimate or approximate the actual temperature of the indicator introduced to the flow in the conduit 8 and use this new (estimated) value in the equation to calculate the flow rate in the conduit.

Therefore, the present disclosure provides for predicting Tᵢₙⱼ based on, or corresponding to a thermal characteristic of the indicator sensor 26, as employed in the catheter 10, and particularly a time constant of the indicator sensor.

Referring to FIG. 5, it is recognized that that different catheters 10 have different time constants based on thermal properties of the specific catheter. The time constant can be measured by multiple methods not limited to the example shown here. For example, the time constant can be calculated by transferring the catheter 10 with the indicator sensor 26 from a constant warmer temperature to a constant colder temperature. The selected time constant can occur at any fraction of the curve. That is, the time constant can be any of a variety of increments, such as but not limited to 64^{ths}, 32^{nds}, 16^{ths}, 8^{ths}, thirds, or halves depending on the desired resolution. Finding or selecting a time constant can occur at any fraction of the resulting temperature curve as the catheter 10 or indicator sensor 26 transitions from the first temperature to the second temperature. Once this time constant is determined, for example in FIG. 6, the time constant can be used to predict or estimate the actual injection temperature Tᵢₙⱼ in clinical measurement.

As seen in FIG. 6, this calibration method for finding the time constant includes imparting a quick and large temperature change to the catheter (and the indicator sensor) and then allowing the temperature of the catheter (and indicator sensor) to stabilize to Tᵢₙⱼ. From the resulting temperature curve during this change, any of a variety of time constants can be selected. For example, in FIG. 6, t_{tc}=the time from the start of the downslope to the time when the temperature curve reaches half of its minimum, or Tₛₕ-(Tₛₕ-Tᵢₙⱼ)/2.

Once the time constant is determined, the time constant can be used to find, estimate, or predict the actual indicator temperature in clinical measurement, as seen in FIG. 5. In the clinical application of FIG. 5, once t_{tc} is known, then t_{tc} is used to prognose, estimate, find, or predict, the temperature of any introduced indicator passing the indicator sensor 26. Thus, Tᵢₙⱼ is the prognosed, estimated, found, or predicted indicator temperature of the introduced indicator (or injection) by this approach. That is, the Tᵢₙⱼ of FIG. 5 is predicted from the temperature at the identified time constant.

Specifically referring to FIG. 6, ΔT_{tc}—the difference in temperature between Tₛₕ and the temperature registered at t_{tc}, thus Tᵢₙⱼ=Tₛₕ−2* ΔT_{tc}. Thus, the unknown Tᵢₙⱼ in the equations above can be predicted through the use of a time constant associated with or representative of the given catheter 10.

Therefore, in one configuration of the present system, a thermal characteristic of the catheter 10 including the associated indicator sensor 26, is determined prior to clinical use of the catheter, wherein the thermal characteristic is used to predict or estimate Tᵢₙⱼ, which in turn is used to calculate the flow rate in the conduit, Q, such as the blood flow rate.

There are different ways to extrapolate the value of Tᵢₙⱼ from the shape of the dilution curve from the indicator sensor 26. The extrapolation may be based on the obtained curve from the indicator sensor 26, or bench testing including the given catheter 10 or the class of the catheter, as well as combinations of the obtained curve and the bench test data. For example, one extrapolation can be based on an exponential approximation of the downslope of the initial temperature decrease from Tₛₕ to Tₘᵢₙ, or part of it. Another extrapolation can be based on multiple bench experiments, where the shape of the injection, the lengths of injection, the volume of injection, and the temperature gradient between T_{b}, Tₛₕ and Tᵢₙⱼ will be varied to produce an extrapolation for Tᵢₙⱼ from various factors, including different types of the introducer sheath and different time constant of indicator sensors. It is understood that different catheters 10 may have different time constants based on their thermal properties. In addition, the bench test data can include different positions of the indicator sensor 26 in both the extracorporeal or the intracorporeal locations in the introducer sheath 80 (FIG. 2 and FIG. 3) It is recognized that the temperature change from Tₛₕ to Tᵢₙⱼ depends on a thermal property or characteristic of the indicator sensor 26, the catheter 10, or both the sensor and the catheter. (FIG. 6) The thermal property is a temperature response of the indicator sensor 26, the catheter 10, or the sensor and the catheter to a given thermal condition or environment or change in the thermal condition or environment. For example, one thermal property can be the time constant of the indicator sensor 26, the catheter 10, or the sensor and the catheter corresponding to the reaction to the thermal change.

Thus, it is recognized that a temperature change from Tₛₕ to Tᵢₙⱼ is dependent on thermal characteristics such as a time constant of the sensor, such as the indicator sensor 26, wherein the sensor can include but is not limited to a thermistor (or other temperature measured means) imbedded in the catheter body 12 and the temperature gradient between Tₛₕ and the Tᵢₙⱼ.

This dependency illustrates the importance of bench calibrations or tests to provide data or characteristics of the catheter 10 (or at least the indicator sensor 26) prior to providing clinical shunt flow measurements with the catheter.

It is also recognized that the introduction of the indicator to the flow rate to be calculated, such as injecting the indicator into the flow in the conduit 8 and for example introducing room temperature saline to the flow in the conduit, will add extra (the indicator) flow into the existing conduit flow (such as the blood flow) and the total flow in the conduit will be a combination of the initial flow in the conduit (such as the blood flow) and the introduced indicator flow. As an example, if the initial flow rate in conduit (the flow to be measured), Qᵢₙ, is 300 ml/min and an injection of indicator such as 10 ml of saline is performed in 3 seconds or 0.05 min, the injection flow rate, Qᵢₙⱼ, becomes 10 ml/0.05 min or 200 ml/min. This new injection flow rate will change the initial sensed flow rate in the conduit 8. Depending on distribution of resistances in the conduit 8 such as hemodynamic resistances, this extra (indicator) flow rate may add to the existing (blood) flow rate. If the main resistance is upstream of the introduction of the indicator, then the total flow Qₜ during the introduction will Qₜ≈Qᵢₙ+Qᵢₙⱼ=500 ml/min. It may not change the initial flow if the main resistance is upstream from the location of the injection in the conduit. The thermodilution principle measures the conduit (blood) flow where the indicator and the conduit (blood) flow are mixed, Qₜ. Therefore, in this scenario, the indicator may mix with the existing flow in the conduit 8 and thus the controller will measure a conduit (or blood) flow rate in a range from 300 ml/min to 500 ml/min depending on distribution of resistances. Assuming that hemodynamic resistances upstream and downstream in the conduit are equal, to minimize the error of initial flow measurement in Eq. 4, half of the introduced indicator flow rate is subtracted from the results, for example, at least the portion of the dilution curve generated during the passing of the indicator into the conduit. Plugging this equation into the current example, the value of 200 ml/min/2, or 100 ml/min, will be subtracted, meaning the error generated by the introduced indicator flow rate is two times smaller and within ±100 ml/min. In prior practice, the introduction, such as the injection, performed through a catheter lumen, the injection flow rate is limited due to a high resistance to the injection because of the long catheter length and the small diameter of the injection lumen. The duration or time of the injection is expected to be in the range of 3-5 seconds. Thus, a 10 ml introduction, or injection, of the indicator, over 3-5 seconds, will give a Qᵢₙⱼ between 120-200 ml/min and thus producing error within ±60 to ±100 ml/min—which is relatively low when compared to the range of the conduit flow rate such as the blood flow rate in the conduit in for example an AV hemodialysis shunt which is 300-4000 ml/min or larger.

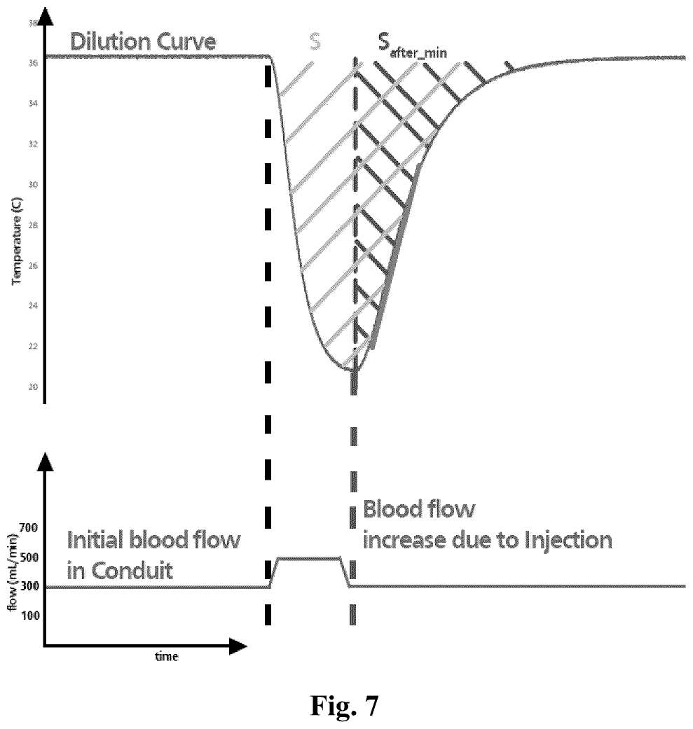

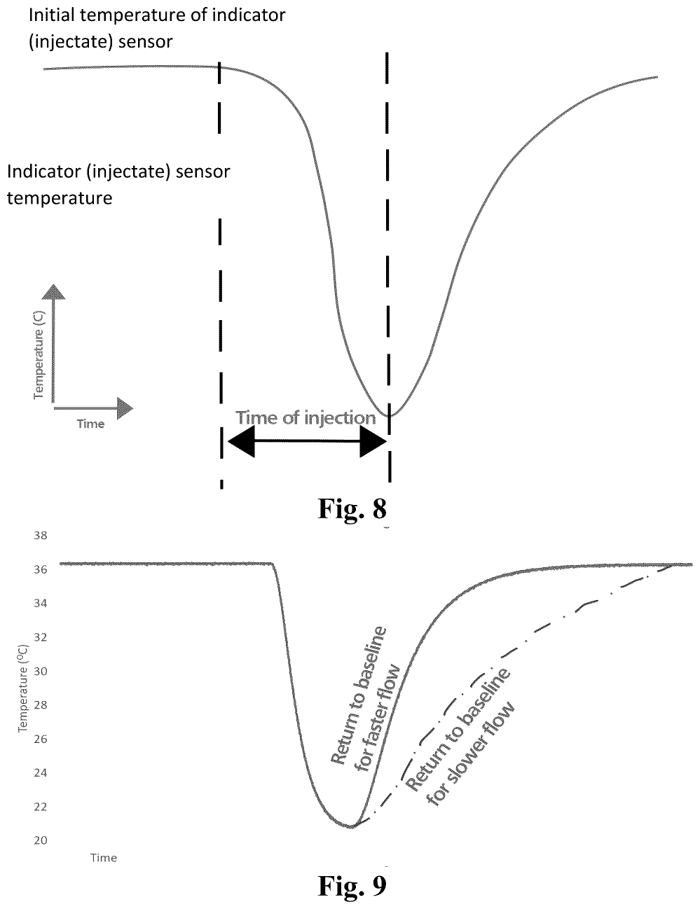

In the case of introduction (or injection) into the side arm port 90 of introducer sheath 80, there are effectively no resistance limitations to the user as the priming volume 23 between the catheter outside diameter and inner surface of the introducer sheath wall may be large and the injection can be done much faster than through the conventional catheter lumen. It is recognized in this case, where introductions, injections, are faster, the error, ±Qᵢₙⱼ/2, can be so large that it can jeopardize the accuracy of the measurement of the flow rate in the conduit. In one configuration, the introduction, or injection, the flow rate, Qᵢₙⱼ, is estimated from the known volume of the indicator, or injection, divided by the time of injection (the introduction period). The time of injection, or the introduction period, can be estimated from the width or downslope shape of the dilution curve (FIG. 7) or from the shape of the temperature change like from downslope time of the indicator sensor (FIG. 8).

As seen in the FIGS. 7 and 8, the dilution curve is generally measured concurrently with the introduction period during when the indicator is introduced into the flow to be calculated, wherein the dilution curve has an apex. Depending on the type of indicator, such as being cooler or warmer than the flow in the conduit 8, the apex may be a maximum or a minimum of the dilution curve. From the start of the dilution curve (generally coinciding with the start of the introduction period to the apex of the dilution curve corresponds to the duration of the introduction period. That is, the dilution curve transitions from the apex when the indicator is no longer being introduced to the flow in the conduit. Thus, a first portion of the dilution curve is generated and measured during the introduction period and a second portion of the dilution curve is generated and measured after the introduction period (after the introduction of the indicator has terminated). The first portion and the second portion transition at the apex of the dilution curve, as there is no additional introduction of the indicator to increase the dilution curve. Alternatively stated, a portion of the dilution curve is generated and identified after the introduction period (after termination of introducing the indicator into the flow to be calculated). Identifying the portion of the dilution curve created after the termination of the introduction of the indicator can be taken as that portion of the dilution curve after the apex. It is understood the apex may not be a singularity, and thus the portion of the dilution curve after termination of introducing the indicator can include the apex. In a further configuration, the portion of the dilution curve after termination of introducing the indicator can include a standard deviation prior to the apex, or 20% or less of the of the dilution curve in standardized normal distribution prior to the apex. It is also contemplated, the portion of the dilution curve after termination of introducing the indicator can exclude a standard deviation after the apex, or 20% or less of the of the dilution curve in standardized normal distribution after the apex. The specific treatment of the apex in determining the portion of the dilution curve after termination of introducing the indicator can be adjusted by bench data as well as clinical data, wherein the controller is configured to accommodate these adjustments to the portion of the dilution curve after termination of introducing the indicator.

Thus, it is noted that the dilution curve can be taken as defining two parts: (i) a first portion, or subspace of the dilution curve spans from the start of the introduction period, or injection, to the apex of the dilution curve (or S_{before_apex}), such as in the example shown, the downslope part or portion which spans from the start of the introduction of the indicator into the flow to be calculated until the curve minimum, or apex, (in the case of saline below body temperature) (FIG. 7) and (ii) an a second portion, or subspace of the dilution curve spans from the apex to a return, or within a given distance, of the dilution curve to its baseline, (in FIG. 7, including the upslope part or portion, after the apex), where the temperature is increasing and the introduction (injection or delivery of cold saline) has terminated. The dilution curve is generated and measured in the conduit simultaneous with a portion of the time during which the indicator is being introduced to the flow in the conduit 8 (the introduction period) as well as a period after the introduction of the indicator into the flow has ceased. For example, the dilution sensor 28 is sufficiently proximal to the distal end of the introducer sheath and the flow rate in the conduit 8 is sufficient that during the introduction period (the time period when the indicator is being introduced into the flow in the conduit), the first portion of the resulting dilution curve in the conduit is being measured. This means that the second part, or subspace of the dilution curve takes place where only the initial conduit flow (blood flow) is present without the effect of the introduced indicator flow. Therefore, the dilution area S_{after_min} (or S_{after_apex}) subspace and the return slope of the dilution curve (or upslope when using an indicator below the temperature of the flow to be measured in the conduit) SL_{after_min} of the dilution curve after the apex represents the initial flow (the flow to be measured) only, and thus the results of the conduit flow rate calculations do not require any adjustments for the flow rate added by the introduced indicator. As set forth in describing the dilution curve before and after the apex, as the dilution curve is a measure of the respective characteristic, property, or parameter of the liquid, collectively referred to as a characteristic. This characteristic change includes changes that are proportional or correspond to the liquid for example, if the liquid characteristics are sensed, the optical, electrical, thermal, or material aspects may be sensed, including but not limited to the electrical conductivity, optical transmissivity, or temperature, velocity of sound, or Doppler frequency. The measure of the characteristic is taken over a period of time, thus “before” the apex means the time before the appearance or measure of the apex, and “after” means the time after the appearance or measure of the apex. Similarly, “before” the introduction of the indicator means the time before the introduction of the indicator into the flow to be calculated and “after” the introduction of the indicator means the time after the introduction of the indicator has ceased. Thus, “from the injection start to the apex” means the time from the start of the indicator introduction to when the apex is measured or observed.

The equations that use the area under dilution curve after the apex, or minimum in the present example, will be analogous to Eq. 3 and Eq. 4. The coefficient K_{after_min} can be produced experimentally, such as for example from bench experiments.

$$Q = \frac{( {T}_{b} - {T}_{inj} ) k * {V}_{inj}}{{K}_{after \_ min} * {S}_{after \_ min}} , \qquad Eq . 5$$

Since the effect of injection flow is negligible, using Eq. 5 and Eq. 4:

$$Q = \frac{[ ( {T}_{b} - ( {T}_{sh} - Δ {T}_{min} - Δ {T}_{ap} ) ] k * ( {V}_{inj} - {V}_{pr} ) + [ ( {T}_{b} - {T}_{sh} ) ] k * {V}_{pr}}{{K}_{after \_ min} * {S}_{after \_ min}} , \qquad Eq . 6$$

Thus, the flow rate in the conduit 8 can be calculated by introducing, during the injection period, the indicator into the flow in the conduit; measuring a resulting dilution curve in the flow to be calculated, the resulting dilution curve extending from during the injection period to after the injection period; and calculating the flow rate of the flow in the conduit from a subspace of the dilution curve, the subspace being a portion of the dilution curve after the injection period, wherein the dilution curve includes the apex and the subspace is after the apex. Thus, the controller can identify in the measured dilution curve a portion of the dilution curve created or generated after termination of introducing the indicator into the flow in the conduit. The controller 60 can then calculate the flow rate corresponding to only the identified portion of the dilution curve.

Another way to calculate the flow rate in the conduit 8 while using the after apex (or after min in the present example) portion of the dilution curve is calculating a slope of the dilution curve after the apex portion of the dilution curve based on the fact that the rate of warming up of the cooled dilution sensor 28 is proportional (related) to the flow rate in the conduit (or the blood flow rate in the conduit). The larger the flow in the conduit 8 (the blood flow rate), the faster the dilution sensor 28 will return to the baseline measure (such as the thermal sensor will be warmed up as shown in FIG. 9). If F(t), for example, where the exponent represents the function of the slope of the dilution curve after the apex (in the present example, the upslope of the after min (after apex) portion of the dilution curve from the dilution sensor 28, then the flow rate, Q, can be represented by:

$$Q = \frac{{K}_{slope}}{T [ F ( t ) ]} \qquad Eq . 7$$

Where Kₛₗₒₚₑ is a coefficient that can be determined experimentally; T[F(t)] is the time constant that represents the slope of the dilution curve after the apex, such as in the present example, the increase of the temperature as the upslope part of the dilution curve after the apex. Thus, the slope of at least a part of the dilution curve identified as after the termination of the introduction of the indicator to the flow can be used.

In operation, the introducer sheath 80 is inserted through a small incision in the skin and advanced into the vein or artery typically using a guidewire. Once the introducer sheath 80 is in place, the guidewire is removed, and the catheter 10 is inserted through the introducer sheath.

Specifically, the catheter 10 is inserted into the introducer sheath 80 and into the A-V shunt until the seating indicia 20 aligns with a predetermined portion of the introducer sheath. For example, the visible print or mark on the catheter body 12 can line up with an entry point of the introducer sheath 80. The indicator is injected into the side arm port 90 of the introducer sheath 80 and passes along the introducer sheath in the priming volume 23, the annular cylinder between the outside the catheter body 12 and the interior surface of the introducer sheath, until the indicator passes from the distal end 84 of the introducer sheath where the indicator mixes with the flow in the conduit 8 to be calculated, such as flowing blood.

In clinical use, referring to FIG. 2, the indicator dilution catheter 10 is placed in the direction of the blood flow pointing downstream (antegrade orientation). In this case, the injected indicator is administered through the introducer side arm 90 of the introducer sheath 80 and enters the A-V shunt at the distal end of the introducer sheath. The indicator passes over the indicator sensor 26 in the extracorporeal region of the introducer sheath, wherein the indicator sensor 26 measures the initial temperature that will be the basis of the approximation/estimation by the controller (software). The indicator dilution temperature change is recorded by the sensor, such as the dilution sensor 28 that is beyond the distal end 84 of the introducer sheath 80 and typically at or near the distal end 14 of the catheter 10 and thus located in the flow to be measured.

Referring to FIG. 3, the indicator dilution catheter 10 is placed in the direction of the blood flow pointing downstream (antegrade orientation). In this case, the introduced indicator (injectate) is administered through the introducer side arm 90 and enters the A-V shunt at the distal end of the introducer sheath 80. The indicator passes over the indicator sensor 26 in the intravascular portion of the introducer sheath 80, wherein the indicator sensor measures the initial temperature that will be the basis of the estimated or approximated injection temperature by the controller, as set forth above. The dilution curve in the flow to be calculated is recorded by the dilution sensor 28 that is beyond the distal end 84 of the introducer sheath 80 and typically at or near the distal end 14 of the catheter 10.

The introduced indicator (injectate) can be a solution that is non-detrimental to the patient, the blood of the patient, or any blood components, and is non-reactive with the system. In one configuration, the indicator is a solution such as isotonic saline and dextrose, or other isotonic solution with a temperature less than blood temperature. In one configuration, the indicator is at room temperature. Although the present disclosure is set forth in terms of a reduced temperature indicator, that is, an indicator with a temperature below blood temperature, it is understood an indicator having a temperature higher than the blood temperature can be used.

Thus, the controller 60 receives the signal from the indicator sensor and the dilution sensor, and obtains the T_{b}; Tₛₕ. The time of the injection (the injection period) can be determined by the controller from the width of the dilution curve. The controller 60 determines S and is provided with the constant K, as well as the priming volume 23. The volume of the injection can be provided by the operator, or a predetermined volume is used in accordance with the controller. The value of AT ap is determined as set forth above.

Various exemplary embodiments of the present disclosure are set forth in terms of medical catheters 10 for the administration of fluids, such as withdrawal from and introduction to the body of a patient and, more particularly, in terms of catheters for vascular access. Vascular access catheters 10 include, for example, central venous catheters, acute dialysis catheters, chronic dialysis catheters, and peripheral catheters. However, it is envisioned that the principles of the present disclosure are equally applicable to a range of catheter applications including surgical, diagnostic, and related treatments of diseases and body ailments of a patient. It is further envisioned that the principles relating to the presently disclosed catheter assemblies 10 may be equally applicable to a variety of catheter related procedures, such as, for example, hemodialysis, cardiac, abdominal, urinary, and intestinal procedures, in chronic and acute applications. Moreover, the presently disclosed catheter assemblies 10 can be used for administration and removal of fluids such as, for example, medication, saline, bodily fluids, blood and urine.

Thus, the present disclosure provides an assembly for introduction of an indicator into the flow to be measured by passing the indicator through a passage in an introducer sheath 80, with the indicator passing along the exterior surface of the catheter 10, such as in the priming volume 23 between the exterior surface of the catheter and the interior surface of the introducer sheath.

In addition, the present disclosure addresses an inability of the indicator sensor 26 to reach the actual temperature of the indicator during the course of the injection. It is contemplated that the actual temperature of the indicator can be estimated through extrapolation or estimation based on the obtained curve from the indicator (injectate) sensor, and particularly the shape of the obtained curve. As set forth above, a thermal characteristic of the catheter 10 such as a time constant of the catheter can be determined prior to clinical use of the catheter and the time constant employed during clinical use to determine the flow rate in the conduit.

The present disclosure also provides alternative methods for analyzing the dilution curve for pertinent information related to the flow in the conduit 8 (such as the blood flow) by (i) the area under the dilution curve, (ii) the area under the dilution curve after the apex of the dilution curve or after the introduction period, and (iii) the slope of the dilution curve after the apex or after the introduction period, as the dilution curve returns to baseline, or to within a given distance to baseline. This analysis has particular applicability to systems where at least a portion of the dilution curve is measured contemporaneously with the introduction period of the indicator. Thus, the analysis can also be applied to when the indicator is introduced into the flow through a catheter, and specifically a catheter lumen.

The present disclosure also provides for increased accuracy by (i) considering the volume of fluid in priming volume 23 in the introducer sheath 80 that has a different temperature than the indicator, but also enters the blood stream, as well as (ii) the portion of the indicator that enters the priming volume but does not pass into the flow to be measured.

The present disclosure also contemplates seating indicia 20 between the introducer sheath 80 and the catheter 10 to assist in operably aligning the catheter and the introducer sheath to reproducibly locate the indicator sensor 26 relative to the introducer sheath.

Although the family of systems are set forth in terms of a thermodilution catheter 10, it is understood the principles and teachings can be used for any dilution catheter. Thus, the application need not be limited only to A-V shunts, but can be employed in any vessel, conduit or channel, where the location of flow resistance is unknown. The flow measurement can be made using any indicator dilution method without departing from this disclosure.

In one configuration, the catheter 10 is configured to cooperatively engage the introducer sheath 80 which has the introducer sheath proximal end 86, the introducer sheath distal end 84, and the passage extending from the introducer sheath proximal end to the introducer sheath distal end, wherein the catheter includes the elongate catheter body 12 having the distal section 14, the proximal section 12, and the at least one lumen 18 extending from proximal section to the distal section, the catheter body including the intravascular portion encompassing the distal section and the extravascular portion encompassing the proximal portion, wherein operable engagement of the catheter body and the introducer sheath locates the distal section of the catheter body beyond the introducer sheath distal end; the first sensor 26 on the catheter body exposed to an exterior of the catheter body; and the second sensor 28 on the distal section of the catheter body. It is contemplated the first sensor 26 is the first dilution sensor and the second sensor 28 is the second dilution sensor. The controller 60 can be operably connected to the catheter 10, wherein the controller is connected to the first sensor 26 on the catheter body 12, wherein the controller is configured to extrapolate the temperature of an indicator passing the first sensor based at least in part on data from the first sensor corresponding to the indicator passing the first sensor. It is contemplated the first sensor 26 and the second indicator sensor 28 are thermal sensors. In one configuration, the elongate catheter body 12 is sized to be axially translatable within the passage of the introducer sheath 80. The seating indicia 20 are configured to indicate the axial alignment of the catheter body 12 and the introducer sheath 80 upon operable engagement of the catheter body and the introducer sheath. Further, the controller 60 can be connected to the first sensor 26 on the catheter body 12, wherein the controller is configured to extrapolate a temperature of the indicator passing the first sensor based at least in part on a thermal characteristic of the first sensor and data from the first sensor corresponding to the indicator passing the first sensor. Specifically, the controller 60 includes or determines the time constant associated with at least the first sensor. Further, at least the first sensor 26 is associated with the given thermal characteristic, such that the thermal characteristic is a time constant.

In one configuration, the catheter assembly is configured for cooperatively engaging the introducer sheath 80, the introducer sheath having the introducer sheath proximal end 86, the introducer sheath distal end 84 having the distal port, and the extravascular introduction port such as the side arm port 90, the catheter assembly 10 including the elongate catheter body 12 having the distal end 14 and the proximal end 12, the catheter body configured to cooperatively engage the introducer sheath to locate the first length of the catheter body within the introducer sheath and the second length of the catheter body extending beyond the distal port 85; the indicator sensor 26 exposed to the external surface of the catheter body within the first length of the catheter body; and the dilution sensor 28 located on the second length of the catheter body. The controller 60 can be operably connected to the indicator sensor 26, wherein the controller is configured to adjust a parameter measured by the indicator sensor. The controller 60 can be operably connected to the indicator sensor 26, and configured to predict the indicator temperature corresponding to a temperature from the indicator sensor and a predetermined time constant associated with at least the indicator sensor. A portion of the catheter body 12 can be sized to pass through the distal port 85, and the distal port and catheter body are configured to pass indicator between an external surface of the catheter body and an interior surface of the introducer sheath 80. Thus, the catheter body 12 is sized to be axially translatable within and relative to the introducer sheath 80.

In a further configuration, the present system includes the introducer sheath 80 having the proximal section 86, the distal section 84, and the passage extending from the proximal section to the distal section; the elongate catheter body 12 sized to be received within the passage and operably coupled to the introducer sheath to locate the first length of the catheter body within the passage and the second length of the catheter body extending from the distal end of the introducer sheath; the indicator sensor 26 on the first length of the catheter body; and the dilution sensor 28 on the second length of the catheter body. In this configuration, the passage terminates at the distal port 85 in the distal section and the catheter body passes through the distal port and the second length of the catheter body passes through distal port to define an indicator flow path between an exterior of the catheter body and the periphery of the distal port.

The present apparatus can include the introducer sheath 80 having the proximal section, the distal section, and the passage extending from the proximal section to the distal section; the elongate catheter body 12 having the distal end 14 and the proximal end 12, the catheter body configured to cooperatively engage the introducer sheath to locate an indicator sensor 26 on the first length of the catheter body within the introducer sheath and the dilution sensor 28 on a second length of the catheter body beyond the distal port 85, and the controller 60 configured to receive signals from the indicator sensor and estimate or extrapolate a characteristic of a passing indicator corresponding to the received signals from the indicator sensor, wherein the characteristic of the indicator is a temperature of the indicator. The controller 60 can be configured to estimate the characteristic of the indicator corresponding to a shape of a dilution curve sensed by the indicator sensor. The controller 60 can also be configured to estimate the characteristic of the indicator corresponding to the slope of the measured curve shape of the dilution curve sensed by the indicator sensor from the condition prior to passage of the indicator and maximum deviation from the condition prior to passage of the indicator. The controller 60 can further be configured to estimate the parameter of the indicator corresponding to the lookup table. The controller 60 can be configured to estimate the parameter of the indicator corresponding to at least one of a length of the injection, the volume of the indicator introduced to the flow to be measured. The controller 60 can be configured to predict the temperature of the passing indicator corresponding to a predetermined time constant associated with the indicator sensor. The controller 60 can be further configured to receive signals from the indicator sensor 26 and estimate or extrapolate a characteristic of a temperature of the passing indicator corresponding to the predetermined thermal property of at least the indicator sensor.

The present apparatus includes the introducer sheath 80 having the proximal section, the distal section, the passage extending from the proximal section to the distal section, and the inlet port 90 configured to introduce an indicator into the passage; the elongate catheter body 12 having the distal end 14 and the proximal end 16, the catheter body configured to cooperatively engage the introducer sheath to locate the first length of the catheter body within the introducer sheath and the second length of the catheter body beyond the distal port, the catheter body sized to define the priming volume between an exterior of the first length of the catheter body and an interior wall of the passage, and the controller 60 configured to calculate a flow rate in the conduit 8, wherein the calculated flow rate at least partly corresponds to a characteristic of one of the indicator or the fluid in the priming volume.

In a further configuration, the apparatus includes the introducer sheath 80 having the proximal section, the distal section, and the passage extending from the proximal section to the distal section; and the elongate catheter body 12 having the distal end 14 and the proximal end 16, the catheter body configured to cooperatively engage the introducer sheath to locate the first length of the catheter body within the introducer sheath and the second length of the catheter body beyond the distal port, wherein at least one of the introducer sheath and the catheter body includes means for longitudinally aligning the catheter body and the introducer sheath, wherein the means for longitudinally aligning includes at least one of a visible mark on the introducer sheath, the catheter body, the detent on at least one of the introducer sheath and the catheter body, the magnet on at least one of the introducer sheath and the catheter body, the detent on at least one of the introducer sheath and the catheter body, the projection on at least one of the introducer sheath and the catheter body.

A method is providing including locating the portion of the introducer sheath 80 within the conduit 8 having the flow rate to be measured, the introducer sheath having the proximal section having the introduction port, the distal section having the distal port, and the passage fluidly connecting the introduction port and the distal port; axially aligning the elongate catheter body with the introducer sheath to locate the first length of the catheter body 12 within the introducer sheath and the second length of the catheter body beyond the distal port; and introducing the indicator into the passage through the introduction port to pass the indicator between the exterior of the first length of the catheter body and the introducer sheath and through the distal port. The method further includes predicting, by the controller 60, the parameter of the introduced indicator corresponding to a predetermined thermal property of the indicator sensor. The method also includes predicting the temperature of the introduced indicator corresponding to the predetermined time constant of the indicator sensor 26 and the sensed temperature from the indicator sensor.

An additional method is provided including providing the introducer sheath 80 having the proximal section including the introduction port, the distal section including the distal port 85, and the passage fluidly connecting the introduction port and the distal port; and axially aligning the elongate catheter body within the passage in the introducer sheath to locate the first length of the catheter body within the introducer sheath and the second length of the catheter body beyond the distal port. The method can further include introducing the indicator into the passage through the introduction port 90 to pass the indicator between the exterior of the first length of the catheter body 12 and the introducer sheath 80 and through the distal port 85.

A method is provided for calculating the flow rate in the conduit 8 based on the indicator introduced into the flow, the method including passing the indicator through the priming volume 23 defined by the exterior of the catheter 10 and the interior of the introducer sheath 80; measuring, by the sensor 28, the dilution curve from the passing of the indicator through the conduit; identifying in the measured dilution curve, the portion of the dilution curve created after termination of the passing of the indicator; and calculating the flow rate in the conduit corresponding to the identified portion of the dilution curve. The method can further include the step of measuring the dilution curve during the passing of the indicator through the conduit. The method can further include the step of calculating the flow rate in the conduit 8 corresponding to one of the priming volume and the predetermined thermal property of at least one of the catheter and the sensor. The method can further include the step of calculating, with the controller 60, the flow rate in the conduit corresponding to the estimate of the temperature of the indicator. The method can further include the step of calculating the flow rate corresponding to the following relationship and analogous relationships:

$$Q = \frac{[ ( {T}_{b} - ( {T}_{sh} - Δ {T}_{min} - Δ {T}_{ap} ) ] k * ( {V}_{inj} - {V}_{pr} ) + [ ( {T}_{b} - {T}_{sh} ) ] k * {V}_{pr}}{{K}_{after \_ min} * {S}_{after \_ min}}$$

where Q is the flow rate in the conduit, T_{b} is an initial temperature of the flow in the conduit, Tₛₕ is the temperature of a primer fluid volume in a priming volume of an introducer sheath prior to the introducing the indicator; ΔTₘᵢₙ is a change in temperature measured at the apex of the dilution curve by the sensor, ΔTₐₚ is an additional temperature change to provide a value for the indicator temperature which is closer to an actual temperature of the indicator, k is a constant, Vᵢₙⱼ is a volume of the indicator, Vₚᵣ is a volume of the indicator that did not enter the flow to be measured, K_{after_min} is a coefficient, and S_{after_min} is an area under the portion of the dilution curve.

The method can further include the step of calculating the flow corresponding to the following relationship and analogous relationships: Q=Kₛₗₒₚₑ/T[F(t)], where Q is the flow rate, Kₛₗₒₚₑ is a coefficient, and T[F(t)] is a time constant that represents a rate of change in a portion of the identified dilution curve. The method can further include the step of measuring the dilution curve during and after the indicator is introduced into the flow.

The present disclosure includes the method of calculating the flow rate in the conduit 8 based on the dilution curve from an indicator introduced into the flow, the method including identifying the apex in the dilution curve in the conduit, wherein the apex defines the first portion of the dilution curve and the second portion of the dilution curve; and calculating the flow corresponding to the second portion of the dilution curve. It is understood the apex is one of a local maximum and a local minimum of the dilution curve. The method further includes measuring an initial temperature of the flow before passing the indicator into the conduit, wherein the first portion of the dilution curve spans from the start of the passing of the indicator into the conduit by the first sensor in the conduit to the apex at the end of the passing of the indicator by the first sensor into the conduit, measuring the temperature of the flow during the passing of the indicator through the conduit; and measuring the temperature of the flow after the passing of the indicator through the conduit, wherein the second portion of the dilution curve spans from the apex until the initial temperature of the flow is reestablished, at least within a predetermined threshold, after the passing of the indicator through the conduit.

The disclosure includes the method of locating the portion of the catheter assembly 10 within the conduit 8 having the flow with a flow rate to be calculated, the catheter assembly having (i) the introducer sheath 80 having an intracorporeal section with an exterior and an interior and an extracorporeal section having the introduction port 90, (ii) a catheter 10 having an exterior, wherein the first sensor 28 is located on the distal end of the catheter, and (iii) the priming volume 23 defined by the exterior of the catheter and the interior of the introducer sheath; and introducing an indicator into the introduction port to pass the indicator within the priming volume.

The disclosure includes the apparatus having the catheter assembly 10 for placement within the conduit 8 having the flow rate to be calculated, the catheter assembly comprising (i) the introducer sheath 80 having the exterior and the interior, (ii) the catheter 10 having an exterior, wherein the first sensor 28 is located on a distal end of the exterior of the catheter, and (iii) the priming volume 23 defined by the exterior of the catheter and the interior of a sheath, the priming volume configured to pass an indicator; and the controller 60 operably connected to the first sensor, the controller configured to calculate a flow in the conduit, wherein the calculated flow at least partly corresponds to a measured dilution curve.

Thus, in contrast prior catheter systems which include a plurality of lumens, an injection port, a radiopaque band, and two thermal sensors. The thermal indicator sensor on these devices is located in or on the catheter and is positioned such that it remains at ambient room temperature prior to an injection. The injection occurs within the catheter, with the main purpose of the sheath being to introduce the catheter to the blood stream. The thermal indicator sensor senses the true value of the indicator, and the dilution thermal sensor records the effect of the indicator on the blood.

While preferred embodiments of the disclosure have been shown and described with particularity, it will be appreciated that various changes and modifications may suggest themselves to one having ordinary skill in the art upon being apprised of the present invention. It is intended to encompass all such changes and modifications as fall within the scope and spirit of the appended claims.

1. An apparatus comprising:
(a) an introducer sheath having a proximal section, a distal section, and a passage extending from the proximal section to the distal section;
(b) an elongate catheter body having a distal section and a proximal end, the catheter body configured to cooperatively engage the introducer sheath to locate a first sensor on a first length of the catheter body beyond the distal port and a second sensor on a second length of the catheter body within the introducer sheath, and
(c) a controller configured to receive signals from the second sensor and estimate or extrapolate a characteristic of a passing indicator corresponding to the received signals from the second sensor.

2. The apparatus of claim 1, wherein the characteristic of the indicator is a temperature of the indicator.

3. The apparatus of claim 1, wherein the controller is configured to estimate or extrapolate the characteristic of the indicator corresponding to a shape of a dilution curve sensed by the second sensor.

4. The apparatus of claim 1, wherein the controller is configured to estimate or extrapolate a characteristic of the indicator corresponding to a downslope of a measured curve shape of a dilution curve sensed by the second sensor from a condition prior to passage of the indicator and maximum deviation from the condition prior to passage of the indicator.

5. The apparatus of claim 1, wherein the controller is configured to estimate or extrapolate the characteristic of the indicator corresponding to a lookup table.

6. The apparatus of claim 1, wherein the controller is configured to estimate or extrapolate the characteristic of the indicator corresponding to at least one of a length of the passing indicator, and a volume of the indicator.

7. The apparatus of claim 1, wherein the controller is configured to predict a temperature of the passing indicator corresponding to a predetermined time constant associated with the second sensor.

8. The apparatus of claim 1, wherein the controller is configured to estimate or extrapolate a temperature of the passing indicator corresponding to a predetermined thermal property of at least the second sensor.

9. The apparatus of claim 1, wherein the controller is configured to receive signals from the second sensor and the first sensor and calculate a flow rate based on the estimation or extrapolation of the characteristic of an indicator passing the second sensor and the signal from first sensor.

10. A method comprising:
(a) locating a portion of a catheter assembly within a conduit having a flow to be measured, the catheter assembly having (i) an introducer sheath having a proximal section having an introduction port and a distal section having a distal port; (ii) a catheter having an exterior, wherein a first sensor is located on a distal section of the exterior of the catheter; and (ii) a passage fluidly connecting the introduction port and the distal port, wherein a second sensor is located within the introducer sheath;
(b) introducing an indicator into the passage through the introduction port;
(c) receiving signals from the second sensor; and
(d) extrapolating or estimating a characteristic of the passing indicator corresponding to the received signals from the second sensor.

11. The method of claim 10, wherein the characteristic of the passing indicator is a temperature of the passing indicator.

12. The method of claim 10, wherein the step of extrapolating or estimating the characteristic of the passing indicator further comprises the step of estimating the characteristic based on a shape of a dilution curve sensed by the second sensor.

13. The method of claim 10, wherein the step of extrapolating or estimating the characteristic of the passing indicator further comprises the step of estimating the characteristic based on a shape of a dilution curve sensed by the second sensor from a condition prior to the passage of the indicator and maximum deviation from the condition prior to the passage of the indictor.

14. The method of claim 10, wherein the step of extrapolating or estimating the characteristic of the passing indicator further comprises the step of estimating the characteristic corresponding to a lookup table.

15. The method of claim 10, wherein the step of extrapolating or estimating the characteristic of the passing indicator further comprises the step of estimating the characteristic corresponding to at least one of a length of the passing of the indicator and a volume of the passed indicator.

16. The method of claim 10, further comprising the step of predicting a temperature of the passing indicator corresponding to a predetermined time constant associated with the second sensor.

17. The method of claim 10, wherein the step of extrapolating or estimating the characteristic of the passing indicator includes the step of extrapolating the characteristic of a temperature of the passing indicator corresponding to a predetermined thermal property of at least the second sensor.

18. The method of claim 10, further comprising the step of calculating a flow rate of the flow based on the extrapolation or estimation of the characteristic of the indicator passing the second sensor and the signal form the first sensor.
